(19) United States
(12) Patent Application Publication
(10) Pub. No.: US 2009/0311778 A1
(43) Pub. Date: Dec. 17, 2009

(54) SUPPLY SYSTEM FOR CELL CULTURE MODULE

(76) Inventors: Rudolph Luning, Gottingen (DE); Uwe Klaus, Radeberg (DE); Laurence J. Purcell, Weston (CA); Stefan Doering, Dresden (DE)

Correspondence Address: BERESKIN AND PARR LLP/S.E.N.C.R.L., s.r.l., 40 KING STREET WEST, BOX 401, TORONTO, ON, M5H 3Y2, CA

(21) Appl. No.: 12/435,611

(22) Filed: May 5, 2009

Related U.S. Application Data
(63) Continuation of application No. PCT/EP2007/009605, filed on Nov. 6, 2007.
(60) Provisional application No. 60/864,678, filed on Nov. 7, 2006.

(51) Int. Cl. C12M 1/14; F04B 19/04

(52) U.S. Cl. 435/299.1; 417/437

(57) ABSTRACT

A device for supplying cell culture modules with nutrients has an arrangement of channels, pumps and valves in or on a plate. The valves may be pinch valves operated by deforming an elastic cover over a solid body and the pump may be a pinch valve pump. The channels may be defined, at least in part, by the plate. The pumps, channels and valves may be located within the thickness of the plate and the cover. The device may be used to supply nutrients to cell culture modules according to a perfusion operation, a re-circulation operation and/or a combination of both. A pump may comprise a generally rigid solid body and a seal. The solid body may wholly or partially define an inlet channel, a plenum and an outlet channel. A port between the inlet channel and the plenum is covered by the seal. A surface of the plenum is deformable. Deforming the surface forces liquid in the plenum to push the seal to cover the inlet channel port and to flow through the outlet channel. When the surface is returned to its original position, fluid flows into the plenum at least partially through the inlet channel displacing or deforming the seal so as to allow flow through the port.

This application is a continuation of PCT/EP2007/009605, filed Nov. 6, 2007, which claims the benefit of U.S. Patent Application No. 60/864,678, filed Nov. 7, 2006, both of which are hereby incorporated herein in their entirety by this reference to them.

### FIELD

This specification relates to devices or processes for cultivating cells or to a pump, conduit or check valve or a method for making a pump, conduit or check valve.

### BACKGROUND

The comments in this background section are not an admission that anything discussed in this section is citable as prior art or part of the common general knowledge of persons skilled in the art in any country.

Some systems for cell cultivation have been developed which provide in some way for the supply of nutrient media to, and the removal of metabolic waste products from, a cell culture. In some systems, cells have been supported on hollow plastic fibers inside of bioreactors. Literature discussing cell cultivation includes the following (1) Sauer, I. M. et al.: The Slide Reactor—a simple hollow fiber based bioreactor suitable for light microscopy; Artificial Organs 29 (3): 264-267, 2005; (2) Sauer, I. M. et al.: Development of a hybrid liver support system. Ann N Y Acad Sd 944: 308-19, (2001); Millis, J. M: et al.: Initial experience with the modified extracorporeal liver-assist device for patients with fulminant hepatic failure: system modifications and clinical impact. Transplantation 74: 1735-46; (2002); and, (4) Glockner, H. et al.: New miniaturized hollow fiber bioreactor for in vivo like cell culture, cell expansion and production of cell-derived products. Biotechnol Prog 17: 828-31 (2001).

PCT Publication No. WO 2004/024303 A2, and related U.S. Publication No. 2006/0014274 A1, disclose a fiber cassette having a housing that is delimited by two congruent base surfaces and at least one circumferential surface and has an interior having at least one cavity. At least one layer of fibers is arranged in the interior of the housing essentially parallel to at least one center plane of the housing, wherein ends of the fibers are anchored fixedly in the interior of the housing. A first one of the at least one cavity defines an outer compartment that surrounds the fibers externally. The at least one center plane does not intersect the base surfaces within the outer compartment. The fibers are arranged U-shaped or essentially parallel to one another and end within the interior of the housing. The housing has at least one opening for supplying and/or removing fluids. PCT Publication No. WO 2004/024303 A2 and U.S. Publication No. 2006/0014274 are incorporated herein in their entirety by this reference to them.

PCT Application No. PCT/CA2006/000739 describes a cell culture bioreactor and is incorporated herein in its entirety by this reference to it.

### SUMMARY

The following summary is intended to introduce the reader to this specification but not to define any invention. Inventions may reside in a combination or sub-combinations of the apparatus elements or process steps described below or in other parts of this document. The invention protected by this document is described in the claims. The inventors do not waive or disclaim their rights to any invention or inventions disclosed in this specification merely by not describing such other invention or inventions in the claims.

The inventors have observed that prior cell cultivation systems involve a complex arrangement of transport hoses, pumps, valves, connectors and other elements for transporting nutrients through a cell culture area. This interferes with economical production of a cell cultivation system, particularly a system of multiple identical culture areas, for example for parallel production to increase output, for drug screening or other applications where it is desirable to grow multiple cultures at the same time. Space requirements of prior systems may also be large.

In an apparatus described herein, a plurality of nutrient or waste transport elements are provided on or inside of, or inside the notional periphery of, a planar transport plate, alternately called a nutrient transport plate. The transport plate is an assembly of elements comprising a solid body and other components collectively adapted to assist in transporting nutrients to, or waste from, a cell culture area or module. The transport plate is planar in the sense that a set of its elements are located within an imaginary plane plus or minus 2 cm. The notional periphery of a transport plate refers to the periphery of a three-dimensional body, for example a parallelepiped, containing the transport plate. A transport plate described in relation to a set of transport elements may still be planar, and have those elements within the notional periphery of the transport plate, despite the presence of other elements attached to the transport plate and extending beyond the notional periphery. Such an apparatus may reduce one or more of the disadvantages of prior nutrient transport systems or at least provide a useful alternative to prior nutrient transport systems or bioreactors.

This specification also describes an apparatus comprising one or more elements formed at least in part by a rigid solid body and arranged for one or more of the supply, removal or recirculation of nutrient media to one or more cell culture modules. The one or more elements for nutrient transport may include one or more of a transport conduit, a valve, a check valve, a connection for a fresh media container or a waste container, a pump, a connection for a cell culture module or an integrated cell culture area. A transport conduit may be formed at least in part by a surface of the solid body. A transport conduit may be a part of a nutrient supply path or a nutrient recirculation path or both. A valve or pump may be formed at least in part by a surface of the solid body. A connection may be attached to the solid body. A valve may comprise a portion of a transport conduit formed at least in part by a flexible body which may be moved into the portion of the transport conduit to prevent or inhibit flow. A pump may comprise a portion of a transport conduit formed at least in part by a flexible body between two valves or check valves. Deflecting the flexible body into the transport conduit portion causes fluid in the portion of the transport conduit portion to move out of the transport conduit portion through one valve and releasing the flexible body causes fluid to flow into the transport conduit portion through the other valve. Portions of a transport conduit that are part of a valve or pump may include a part of a resilient cover attached to the solid body. A transport conduit may have a silicone surface.

An apparatus may optionally also include one or more of a sampling connection, a transducer, a sensor mount, a meter, a thermal element or a gassing element. A sensor may be positioned so as to not contact nutrient solution. A connector may be a standard, universal, or frequently used connector to facilitate the integration of an arbitrary cell culture module to the nutrient transport plate. An apparatus may be made of a sterilisable material such as a plastic. This allows an apparatus to be used as a disposable transport system, if the corresponding cell culture module is detachable or also disposable. Metals, glass, ceramics or other materials may also be used. An apparatus may be suitable for, and a process may comprise using an apparatus for, nutrient transport to modules containing or cultivating protozoa, bacteria, yeasts, fungi, plants or cells of vertebrates, for example mammals. An apparatus may be combined with a cell culture module according to WO 2004/024303 A2 or other cell culture modules which may be, for example tubular, planar, rectangular, star-shaped or other shapes.

This specification also describes a process comprising providing an apparatus as described above and using the apparatus, for example by moving the flexible bodies of the apparatus, to transport nutrients through cell culture modules in perfusion, recirculation or a combination of these two operating modes.

This specification also describes a transport plate wherein at least one of a conduit, a valve, a check valve or a pump comprise or essentially consist of a portion of solid body and a portion of a flexible body or cover attached to the solid body.

This specification also describes a check valve or a pump. A pump may comprise a generally rigid solid body and a seal. The solid body may wholly or partially define an inlet channel, a plenum and an outlet channel. A port between the inlet channel and the plenum is covered by the seal. A cover of the plenum is deformable. The seal acts as a pair of check valves. Deforming the cover forces liquid in the plenum to push the seal to cover the inlet channel port and forces liquid to flow through the outlet channel. When the surface is returned to its original position, the seal is displaced or deformed and fluid flows into the plenum through the inlet channel.

### BRIEF DESCRIPTION OF THE DRAWINGS

FIG. 1 is an orthographic projection of the top, side and end of a pump.

FIG. 2 shows a cross sectional elevation view of the pump of FIG. 1 cut along line 2-2 of FIG. 1.

FIG. 3 shows a plan view of the pump of FIG. 1 cut along the line 3-3 of FIG. 1.

FIG. 4 is a cross-sectional end view of the pump of FIG. 1 cut along the line 4-4 of FIG. 2.

FIG. 5 is a cross-sectional elevation view of another pump with an actuator and controller.

FIG. 6 is a top view of a transport plate.

FIG. 7 shows a membrane cell culture module.

FIG. 8 shows an actuator set.

### DETAILED DESCRIPTION

Various apparatuses or processes will be described below to provide an example of an embodiment of each claimed invention. No embodiment described below limits any claimed invention and any claimed invention may cover processes or apparatuses that are not described below. The claimed inventions are not limited to apparatuses or processes having all of the features of any one apparatus or process described below or to features common to multiple or all of the apparatuses described below. It is possible that an apparatus or process described below is not an embodiment of any claimed invention. The applicants, inventors and owners reserve all rights in any invention disclosed in an apparatus or process described below that is not claimed in this document and do not abandon, disclaim or dedicate to the public any such invention by its disclosure in this document.

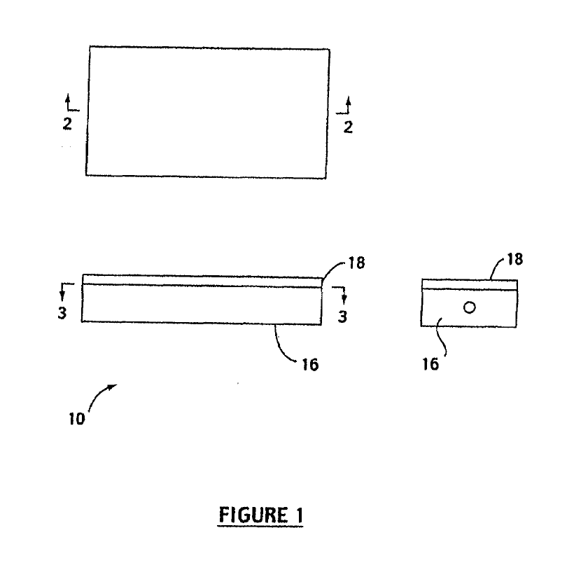

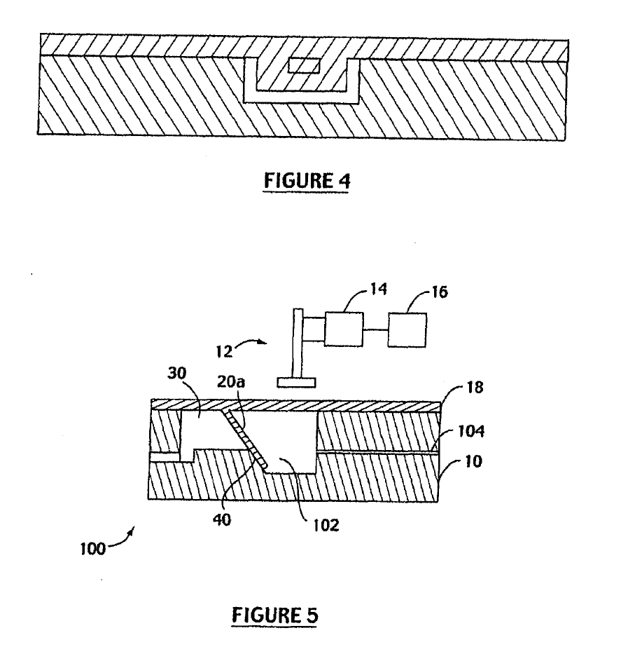

FIGS. 1 to 4 show a pump 10, an actuator 12, a controller 14 and a power supply 16. Pump 10 comprises a rigid body 16 and a resilient body 18. Rigid body 16 may be made, for example, of hard plastic. Resilient body 18 may be made, for example, of silicone if gas transfer is desired or rubber if not. Resilient body 18 may have a planar section 22 and one or more flap seals 20. Planar section 22 is bonded, for example with silicone sealant or glue, to an upper surface 26 of solid body 16. Resilient body 18 thus covers, or provides an upper surface to all or part of various elements completed by solid body 16, such as an inlet channel 30, a plenum 32, an outlet channel 34, an inlet valve body 36 and an outlet valve body 38. Inlet channel 30 communicates with inlet valve body 36 through an inlet channel port 40. Inlet valve body 36 communicates with plenum 32 through inlet passage 42. Plenum 32 communicates with outlet valve body 38 through outlet passage 44 and outlet port 46. Outlet valve body 38 communicates with outlet channel 34. A first flap seal 20a sits, when not acted on by external forces, adjacent a wall of inlet valve body 36 that is pierced by inlet channel port 40. A second flap seal 20b sits, when not acted on by external forces, adjacent a wall of outlet valve body 38 that is pierced by outlet port 46. Flap seals 20 as shown are molded inserts bonded to solid body 16, the rest of resilient body 18 or both. Alternately, flap seals 20 may be integral with resilient body 18. Further alternately, flap seals 20 may be made of a piece of resilient sheet material folded to provide a flap part and a tab for bonding to solid body 16 or resilient body 18.

In operation, a liquid is provided in communication with inlet channel 30 and within the space between rigid body 16 and resilient body 18. Actuator 12, which may be for example a solenoid, is moved downward by controller 14, powered by power supply 16, to press a portion of resilient body 18 into plenum 32. This displaces the liquid in plenum 32 which pushes first flap 20a against the wall of inlet valve body 36 and so at least partially seals, or inhibits liquid flow through, inlet channel port 40. Pressure, or displacement of, liquid in plenum 32 also moves second flap 20b away from outlet port 46. In this way, flaps 20, and valve bodies 36, 38 are or function as one-way valves. Liquid flows from plenum 32 through outlet port 46 to outlet valve body 38 and out of outlet channel 34. When actuator 12 is released, the planar section 22 of resilient member 18 returns to its at rest state. Fresh liquid flows through inlet channel 30 and inlet channel port 40, displaces first flap 20a, and flows into inlet valve body 36 and plenum 32. At the same time, second flap 20b inhibits or prevents flow of liquid from outlet valve body 38 back into plenum 32. Flow of liquid into plenum 32 may be caused by the force or suction pressure of resilient member 18 returning to its original position, or by a static head difference between inlet channel 30 and outlet channel 34 or by both. Reciprocating actuator 12 may move a volume of fluid through pump 10 with each depression of actuator 12.

FIG. 5 shows a second pump 100. In second pump 100, inlet valve body 36 and plenum 32 are replaced by a valve body plenum 102 and inlet passage 42 is deleted. All items downstream of plenum 32 in the pump 10 are replaced in pump 100 by a flow restricting outlet 104. In operation, when actuator 12 presses a part of resilient member 18 into valve body plenum 102, liquid in valve body plenum 102 forces flap 20a to at least partially seal port 40. Liquid from valve body plenum 32 flows through restricting outlet 104 to leave second pump 100. When actuator 12 is released, fluid enters valve body plenum 102 at least partially through inlet channel 30 and around flap 20a, since restricting outlet 104 inhibits the return of liquid to valve body plenum 102.

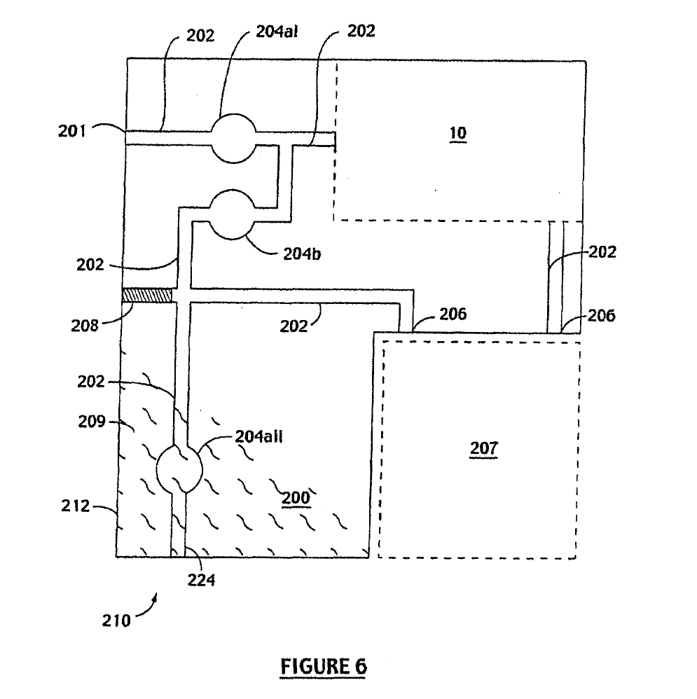

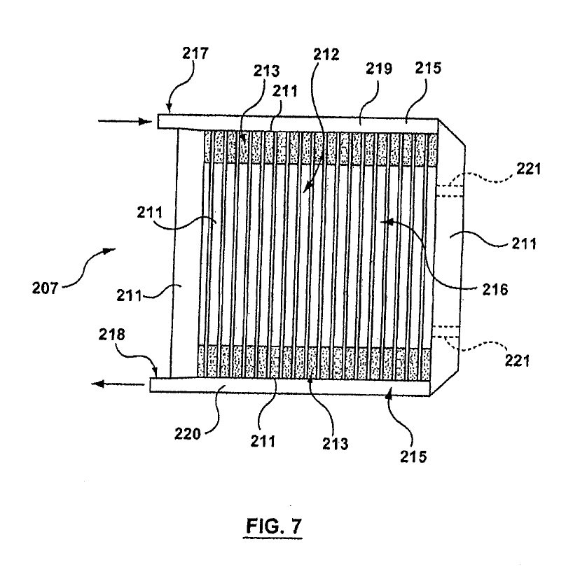

Referring to FIG. 6, a bioreactor 210 comprises a nutrient transport plate 212 and a cell culture module 207. Cell culture module 207 is plugged into, and optionally may be removed from, cell culture module connections 206. Cell culture module connections 206 may be holes or grooves machined in solid body 209 optionally with fittings (not shown) inserted into them. An example of a cell culture module 207 is shown in more detail in FIG. 7. As shown, the cell culture module 207 has a cover removed from it that would otherwise enclose an outer compartment 216 and parts of an inner compartment 215. Inner compartment 215 also includes the lumens of hollow fiber membranes 212. The walls of hollow fiber membranes 212 and potting compound 213 as well as a base structure 211 and the cover (not shown), separate inner compartment 215 from outer compartment 216. Cells may grow on the membranes 212 or otherwise in second compartment 216. Nutrients may be supplied to the cells through the first compartment 215 and the walls of membranes 212. In particular, a nutrient solution can be input into supply port 217, into a first channel 219 portion of first compartment 215, through the lumens of membranes 212, into a second channel 220 portion of first compartment 215 and out through a waste port 218. While traveling through this path, some nutrients, for example carbohydrates or gases, pass through the walls of membranes 212 to be consumed by cells in second compartment 216. Some waste products released by the cells travel from second compartment 216 through the walls of membranes 212 and are carried away with the nutrient solution. Cell culture module 207 is attached to nutrient transport plate 212 by inserting supply port 217 and waste port 218 into cell culture module connections 206. Ports 217 and 218 may be glued into connections 206 for a permanent attachment, or removably sealed together through a press in or other fit. Optionally, cell culture module 207 may have auxiliary ports 221 to allow for adding or removing substances to second compartment 216 without passing through the walls of membranes 212. The auxiliary ports 221 can be used, for example, to extract cells or cell products, secretions, viruses, proteins or low molecular weight substances. The bioreactor 210 can thus be used for a variety of applications including, for example, growing high density cell cultures, testing or screening for the reaction of cell cultures to various substances, or harvesting products made by cells.

Solid body 209 of nutrient transport plate 212 may be made from a sheet of a rigid material, for example a hard plastic, with a thickness in the range of, for example, 3 mm to 10 mm. Solid body 209 may be made by cutting the sheet of material to a selected width and length or perimeter shape, for example in the range of 3 cm to 15 cm. Transport channels 202 and other grooves or depressions may be made, for example by router, in the surface of solid body 209. Alternately, solid body 209 may be formed, for example molded, with the required grooves or depressions. A nutrient supply is connected to the nutrient transport plate 212 through nutrient connector 201 which may comprise a fitting slipped into a transport channel 202 or a hole in rigid body 209. Waste solution may leave transport plate 212 through a waste connector 224 which may be made as described for nutrient connector 201. Samples may be extracted from a sample valve 208 which may comprise, for example, a true valve, an opening with a removable cap or, as shown, a plug of material forming a self sealing septum.

The nutrient transport plate 212 provides two basic flow paths. A first flow path starts at the nutrient connector 201 and ends at waste connector 224 after passing through an area containing waste nutrient, for example the first compartment 215 of cell culture module 207 or an integrated cell culture area or a part of the second flow path described below. A second flow path travels in a loop through the nutrient transport plate 212 from a first cell culture module connection 206 to the other and then through the first compartment 215 of cell culture module 207 back to the first cell culture module connection 206, or through a similar path involving an integrated cell culture area. While nutrient is flowing through the second flow path, a connection to nutrient connector 201 may be left open so that nutrient can be drawn in to replace nutrient consumed by the cells. Between the two flow paths, fresh nutrient solution can flow from a nutrient source to membranes 212, old nutrient solution can flow out to a waste container or drain, or a nutrient solution can re-circulate though the membranes 212. Further, a bioreactor 210 can be operated cyclically with, for example a period of flow through the first flow path, then a period of flow through the second flow path repeated in cycles. Control and propulsion through these flow paths may be provided as described below.

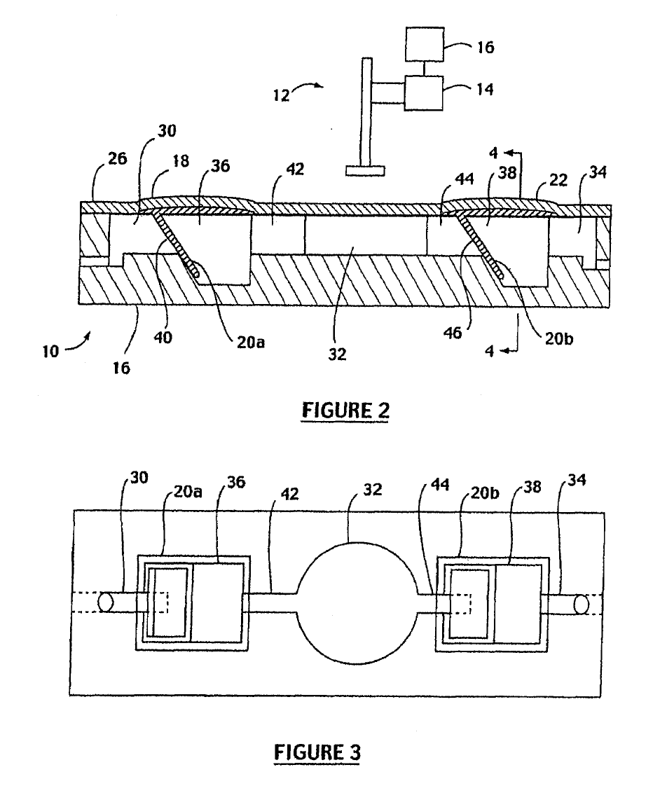

A portion of some or all of transport channels 202 may be part of a valve body 204 formed as described for the plenum 32 of FIGS. 2 and 3. The valve body 204 works with an actuator 12 as described previously. The actuator 12 is operable to push in portion of cover 200 so as to fully or partially close the flow path through valve body 204 and prevent or inhibit nutrient flow and thereby provide a 204 valve. Cover 200, although only a portion is shown in FIG. 6, as represented by the wiggly lines in the lower left corner of the bioreactor 210, covers the entire upper surface of solid body 209 or as much of it as required to enclose valve bodies 204 and cover the otherwise open grooves in the solid body 209 so as to complete transport channels 202. Cover 200 may be made of a resilient material and may further be made of an oxygen permeable material such as silicone. Cover 200 may be bonded, glued, welded or otherwise attached to the upper surface of solid body 209. When valve body 204aii is closed and valve body 204b (and optionally valve body 204aii) is open, the second flow path is provided. When valve body 204b is closed and valve bodies 204a are open, the first flow path is provided. Flow through the first flow path can also be provided by having valve bodies 204ai and 204b closed and valve bodies 204aii open while pump 10 is operated (actuator 12 is moved into plenum 32) as described earlier, closing valve body 204aii and opening valve bodies 204ai and 204b while valve body 204a remains pinched, then removing the actuator 12 from plenum 32 of pump 10. According to that operation, valve bodies 204ai and 204b are operated simultaneously and may be activated by one actuator 12 to be described further below.

Nutrient transport plate 212 has a pump 10 described earlier. When using pump 10 parts of transport channels 202, may swell temporarily to take up a volume of nutrient solution displaced by the pump 10. The bioreactor 210 may also be operated by alternating between the flow paths. For example, pump 10 may be operated one or more times while the first flow path is open to expel old nutrient fluid containing waste products and intake fresh nutrient solution. Thereafter, pump 10 may be operated one or more times to recirculate nutrient fluid. Then these two steps above are repeated so as to cycle back and forth between refreshing and recirculating the nutrient solution. For example, a cycle may comprise operating pump 10 once to refresh nutrient, followed by operating pump 10 for 2 to 10 times to recirculate the nutrient solution. Alternately, an additional check valve may be inserted into nutrient connector 201 to allow nutrient solution to enter into but not exit from nutrient connector 201. Then valve bodies 204ai and 204b may be left open while valve body 204a is occluded but left slightly open. In this configuration, operating pump 10 causes a continuous recirculation but with a continuous bleed of waste and feed of fresh nutrient solutions. Check valves may be optionally be replaced by other valves, for example pinch valves, operated so as to mimic the action of check valves.

Bioreactor 210 may also have various optional additional components for process implementation, monitoring or control. If cover 200 is made of a gas permeable material, for example silicone, it acts as a gassing element. Temperature elements may heat or cool the cell culture module 207. A temperature sensor may be inserted into a hole drilled into the edge of solid body 209 intersecting a transport channel 202. A temperature sensor may measure nutrient solution temperature and may be connected to a temperature element in a control or feedback loop to maintain a desired temperature. One or more optical sensors may be inserted into holes drilled into the edge of solid body 209 so as to be near, but not fluidly connected to a transport channel 202. Optical sensors may comprise a pH sensitive transducer or a dissolved oxygen level sensitive transducer.

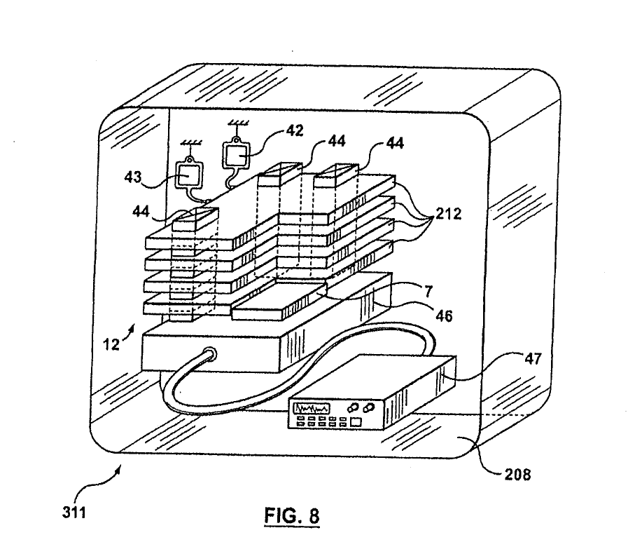

FIG. 8 shows an assembled cell cultivation system 311 having one or more stacked nutrient transport plates 212. Nutrient transport plates 212 are connected in parallel to a nutrient container 42 and a waste container 43. Actuators 12, comprising a base 46 able to produce electric fields in locations below various stacked magnets 44, are situated over the nutrient transport plates so as to be able to deflect covers 200 to provide valve operations and pumping. The system 311 is optionally located within a controlled environment chamber 208. Control of actuators 12 may be linked through a programmable logic controller or other controller 47 to sensors or other controlled devices.

The invention or inventions which are currently claimed in this document are described in the following claims.

We claim:

1. An apparatus for supplying a cell culture module with nutrients comprising, a rigid body having a plurality of inter-connected channels open to a surface of the rigid body, and a resilient body attached to the surface of the solid body covering the channels.

2. An apparatus according to claim 1, wherein the channels comprise a pump body, a transport channel, and a valve body.

3. An apparatus according to claim 1, further comprising connectors for nutrient and waste containers.

4. An apparatus according to claim 1, wherein the channels are configured to allow unidirectional flow through the cell culture module.

5. An apparatus according to claim 1, wherein the channels are configured to allow nutrient re-circulation through the cell culture module.

6. An apparatus according to claim 1, wherein the channels are configured to allow selective re-circulation or unidirectional flow.

7. An apparatus according to claim 1, further comprising a cell culture module having a thickness not greater than the thickness of the solid body and resilient body.

8. An apparatus according to claim 1, further comprising a controller and an actuator.

9. An apparatus comprising a rigid solid body and a flexible cover defining conduits providing one or more of a first flow path between a nutrient connector and a waste connector and a second flow path from one end of, or a connector to, a cell culture area to another end of, or connector to the cell culture area.

10. An apparatus according to claim 9 wherein the cover may be deformed to close a conduit.

11. An apparatus according to claim 9 further comprising a pump comprising a valve upstream and downstream of a flexible body deformable to reduce the interior volume of a conduit between the two valves.

12. An apparatus according to claim 11 wherein the upstream and downstream valves are check valves.

13. An apparatus according to claim 9 having both flow paths configured such that deforming the cover may open one flow path while closing the other.

14. An apparatus according to claim 9 wherein a portion of the first flow path comprises a portion of a conduit that is also a portion of the second flow path.

15. An apparatus according to claim 9 comprising a pinch valve or a pinch valve pump substantially within the thickness of a planar element including the solid body and comprising a portion of the solid body.

16. An apparatus according to claim 9 further comprising an actuator and a controller.

17. An apparatus according to claim 9 further comprising a nutrient supply and a waste container.

18. A pump comprising:
a generally rigid solid body defining a cavity;
a deformable surface covering the cavity to form a plenum;
an inlet channel to the plenum; an outlet channel from the plenum;
a moveable seal between the inlet channel and the plenum,
wherein the elements above are configured such that flow out of the plenum is restricted to a greater extent through the inlet channel than through the outlet channel and flow into the plenum is restricted to a greater extent through the outlet channel than the inlet channel.

19. The pump of claim 18 wherein the movable seal is integral with the deformable surface.

20. The pump of claim 19 wherein the deformable surface comprises silicone.
